(19) United States
(12) Patent Application Publication
(10) Pub. No.: US 2013/0017607 A1
(43) Pub. Date: Jan. 17, 2013

(54) Nanotube carrier substrate for primary tissue culture

(71) Applicants: Stefan Mayr, Leipzig (DE); Mareike Zink, Leipzig (DE); Valentina Dallacasagrande, Leipzig (DE); Josef Käs, Leipzig (DE); Andreas Reichenbach, Leipzig (DE)

(72) Inventors: Stefan Mayr, Leipzig (DE); Mareike Zink, Leipzig (DE); Valentina Dallacasagrande, Leipzig (DE); Josef Käs, Leipzig (DE); Andreas Reichenbach, Leipzig (DE)

(73) Assignee: UNIVERSITAET LEIPZIG, Leipzig (DE)

(21) Appl. No.: 13/547,076

(22) Filed: Jul. 12, 2012

(30) Foreign Application Priority Data
Jul. 13, 2011 (EP) 11 173 854.8

(51) Int. Cl. C12M 3/00; C12N 5/071; B82Y 5/00

(52) U.S. Cl. 435/402; 435/289.1; 435/395; 977/923; 977/746; 977/743; 977/742

(57) ABSTRACT

A carrier substrate for primary tissue culture has a nanotube array. A tissue culture vessel has an outer vessel and a nanotube carrier substrate with a nanotube array, located within the outer vessel, wherein the surface roughness of the nanotube array is 1 nm to 100 nm. The nanotube array is used for in vitro culturing of primary tissue in connection with a tissue culture vessel for in vitro culturing of primary tissue and a method for in vitro culturing primary tissue, wherein a nanotube array is arranged essentially horizontal inside an outer cell culture vessel, so that openings of the nanotubes point at least in upward direction, the nanotube array is contacted with cell culture medium and an isolated primary tissue sample is placed on top-side on said nanotube array.

The present invention relates to a carrier substrate for primary tissue culture comprising a nanotube array. The invention is applicable in biomedical and biophysical research and clinical applications for in vitro culture of explanted tissue.

Organ tissue culture in vitro is essential for studying the biology, chemical or physical properties of a living tissue of interest, as well as the effect of drugs and pharmaceuticals on said tissue and the living cells the tissue comprises. It is to be distinguished between in vitro culturing of explanted tissue, which is an object of the disclosed invention, and tissue grown in vitro to build up new organs for organ transplantations.

Many studies require the culture of an entire tissue freshly explanted from the body (also referred to herein as “organ culture” or “tissue culture”, these terms being used synonymously herein) which reflects in vivo conditions much better. In organ culture parts of an organ or a whole organ are cultures with the aim to maintain the architecture of the tissue and to direct it towards normal development. Hence, it is essential that the tissue is not disrupted or damaged, which requires careful handling. The culture media used for organ culture are generally the same as those used for cell culture. Therefore the terms “cell culture media” and “tissue culture media” are herein used synonymously.

Organ culture using organs derived from adult animals is challenging and often impossible for explanted mammalian organ tissue due to their greater requirement of oxygen. Greater amounts of serum cannot compensate for this need since it causes toxic effects. Therefore, in the art organ culture is performed usually with embryonic cells that are not differentiated yet rather than performing organ culture with adult organ tissue.

In the art, two main alternatives for cultivation of explanted adult organ tissue are provided. Firstly, instead of cultivating complete tissue samples, cells are extracted from the tissue and cultured without the extracellular matrix surrounding the cells in three-dimensional cell culture (Desai 1999, Eschbach 2005, Giselbrecht 2006, Kunz-Schughart 2004, Leclerc 2003, Snyder 2001, Yamauchi 2003). However by using these methods, the entire tissue is only represented to a very small extend and as the extracellular matrix is lacking, the properties of the cultivated tissue cells and the in vivo behavior differ.

Secondly, the entire explanted organ tissue can be cultivated in vitro. For this application, conventional cell culture plastic dishes cannot be employed. Therefore various tissue substrates and alternative carrier substrates or devices were developed that assure continuous feeding of the organ with nutrients and that avoid dissolution of the cultured organ.

Organ culture substrates known in the art are nitrocellulose filters (Germer 1997, Kuhrt 2008), cellulose filters (Caffe 2001), PET membranes such as Millipore Millicell inserts (Ogilvie 1999, Johansson 2005), glass (Xin 2007), perforated grids made of stainless steel (Kaempf 2008) or incorporation into agar gels contained in the cell culture medium (Spencer 2008). Further, known organ cultures are performed as roller cultures (Feigenspan 1993, Rzeczinski 2006) or without a support by fixing the organ between two rings (Kobuch 2008). All substrates except for glass and stainless steel grids are soft and highly susceptible and therefore not applicable for many applications that require mechanically stable substrates. Culturing of adult tissues on robust substrates such as glass or also on stainless steel grids could not be performed for several days yet as the cultured organ dies rapidly due to non-physiological conditions.

Since adult mammalian organ tissue is more challenging to culture compared to embryonic tissue, only short culture periods of up to few days can yet be reached on specific substrates. In vitro culturing of a mature retina on a cellulose-based substrate could be performed for up to six days (Koizumi 2007).

US 2008/0176206 A1 disclosed a substrate for in vitro culturing of cardiovascular tissue. It is comprised of a co-polymeric foam with a bioabsorbable material. The system disclosed in US 2008/0176206 A1 is specifically designed for the regeneration of cardiovascular tissue.

The tissue culture substrates provided in the art are all based of biocompatible organic materials that can be used only once. Further, tissue culture substrates known in the art are not versatile and applicable for a variety of different tissues. Hence, there is a need for universal tissue culture substrates that can be easily designed and optimized for the requirements of different tissues and that allow an in vitro organ culture for several days, especially with explanted adult organ tissue.

In the art, metal oxide based cell culture substrates recently got into focus for different culturing applications such as adherent cell culture. WO 2011/052979 A2 discloses a method for reprogramming somatic cells to induced pluripotent stem cells on a nanotube substrate comprising metal oxides. In the method of WO 2011/052979 A2 transcription factors, such as Oct3/4, Sox2, c-Myc, are introduced into a silicon dioxide layer that is located on the surface of a nanotube substrate thus forcing expression of specific genes in the cultured cells.

It is the object of the invention to provide an improved re-usable tissue culture substrate that can be easily adjusted to the requirements of different adult organ tissue.

The object is solved by providing a tissue culture vessel that is comprised of an outer vessel and a nanotube carrier substrate located within the outer vessel. According to the invention the nanotube carrier substrate comprises a nanotube array, wherein the surface roughness of the nanotube array is 1 nm to 100 nm. The term surface roughness as used herein (and in the art) is defined as the standard deviation of the average nanotube height on the surface of the nanotube array. Preferred nanotube arrays are metal oxide nanotube arrays or carbon nanotube arrays, particularly preferred are metal oxide nanotube arrays.

Another aspect of the invention is the use of a nanotube array for in vitro culturing of primary tissue, preferably primary mammalian tissue, preferably for organ culture.

Another aspect of the invention is the use a tissue culture vessel according to the invention for in vitro culturing of primary tissue, preferably primary mammalian tissue, preferably for organ culture.

Another aspect of the invention is a method for in vitro culturing primary tissue, preferably primary mammalian tissue, wherein
a nanotube array is arranged essentially horizontal inside an outer cell culture vessel, so that openings of the nanotubes point at least in upward direction,
the nanotube array is contacted with cell culture medium,
an isolated primary tissue sample is placed on top-side on said nanotube array.

With a essentially horizontal arrangement of the nanotube array inside the outer cell culture vessel is meant herein that in relation to the bottom of the outer cell culture vessel the array of adjacent nanotubes is arranged horizontally (in a range of 170° to 190°, preferably ±5°), which leads to an essentially vertical (in a range of 80° to 100°, preferably 90°±5°) arrangement of individual nanotubes with respect to the bottom of the outer cell culture vessel.

The inventors have found that explanted primary adult tissue samples can be successfully cultivated in vitro on a titanium dioxide nanotube array for at least 14 days. The explanted tissue samples were therefore placed onto the titanium dioxide nanotube array with cell culture medium fed from the side of the nanotube substrate or, alternatively, from the bottom through the nanotube substrate. Because the nanotube array is wettable by cell culture medium, that is brought in contact with the nanotube array, the latter is drawn into the nanotubes by capillary forces thereby creating a thin film of cell culture medium on the surface of the nanotube array (that is to be distinguished from the bulk liquid of cell culture medium that is filled into the cell culture vessel). The explanted organ itself was not in contact with the cell culture medium bulk liquid but only with the nanotube substrate. The supply of nutrition from the cell culture medium is surprisingly ensured only by the thin film of cell culture medium on the nanotube substrate surface that is continuously restored (by drawing fresh medium onto the substrate by means of capillary forces).

By providing the possibility for culturing explanted organ tissue without direct contact to the bulk liquid of cell culture medium the dissolution of the tissue by cells migrating from the tissue to the liquid or along the nanotube substrate surface is minimized. In-growth of cells from the organ tissue into the nanotube array can be minimized. Thus, with the invention it is possible to provide a tissue culture substrate that allows removal of the cultured organ tissue from the substrate surface without damaging the sensitive tissue structure. However, for some applications it is desired to provide a tissue culture substrate that allows adhesion of the cultured organ tissue, e.g. for measurement of physical parameters of the tissue. This object can also be solved by the invention as by variation of tube diameter or by coating of the nanotube substrate with different bioactive substances to increase tissue adhesion to the nanotube surface.

Advantageously apart from the improved nutrient supply of the organ tissue ensured by the nanotube substrate, the substrate exhibits high mechanical stability (and is thereby easy to handle), is sterilizable and thus reusable for further culturing applications. With methods for varying the properties of nanotube arrays being well known in the art, it is possible to adjust the nanotube substrate properties such as tube diameter, surface roughness and wall thickness easily for any type of organ tissue to be cultured.

Nanotube arrays and methods for their production are known in the art (Roy 2011). A nanotube array comprises a multitude of nanotubes arranged in parallel being adjacent to one another (adjacent individual nanotubes are in contact with each other). The properties of nanotube arrays, such as tube diameter, surface roughness, wall thickness, tube length etc. can be influenced by variation of the processing conditions.

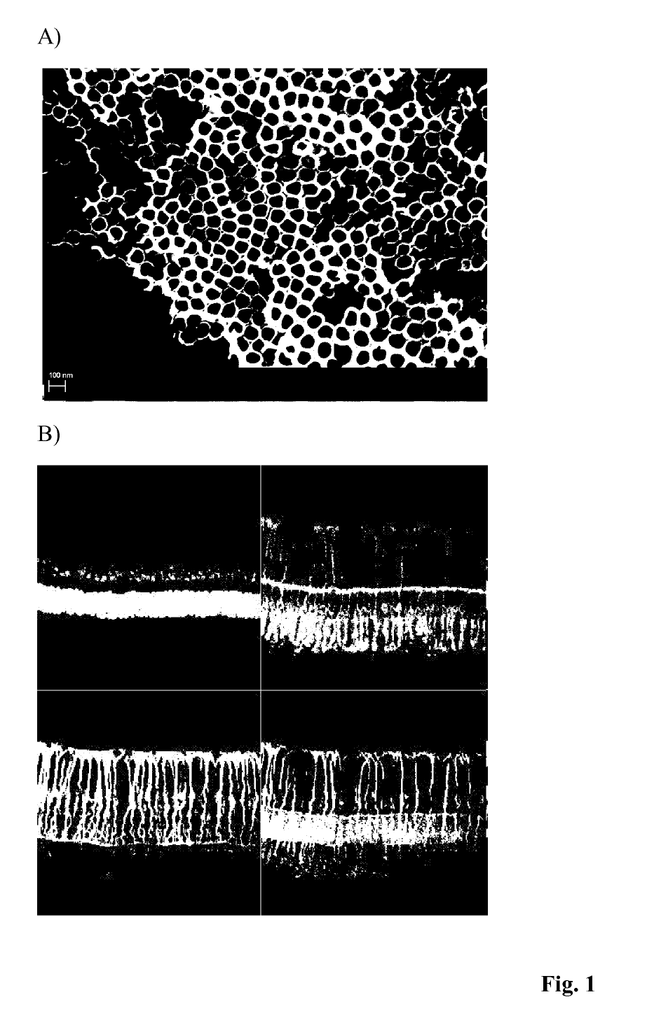

In the invention, metal oxide nanotube arrays or carbon nanotube arrays are preferred. Metal oxide nanotube arrays are commonly formed by electrochemical anodisation of a metallic substrate (e.g. titanium) under a specific set of environmental conditions yielding self aligned nanotube layers of the respective metal oxide (herein referred to as “metal oxide nanotube array”, FIG. 1A, 2A). In the invention, nanotube arrays of metal oxides selected from titanium, zirconium, hafnium, silicon, aluminum, gold, silver, copper, platinum, vanadium, palladium, and niobium, as well as their alloys are preferred, particularly preferred are titanium oxide nanotube arrays, particularly preferred are titanium dioxide nanotube arrays. These nanotubes can reside in a crystalline or amorphous structure, which is particularly preferred.

The average inner diameter of the nanotubes contained in the nanotube array is preferably 10-500 nm, particularly preferred 20-150 nm. The average tube length in the nanotube array is preferably 100 nm-500 μm. The surface roughness of the nanotube array is preferably 1 nm to 100 nm. Nanotube arrays with a surface roughness of 5-100 nm are preferred for cultivation of retinal tissue. Nanotube arrays with a low surface roughness from 1 nm to 10 nm are particularly preferred for cultivation of brain tissue. However, the diameter of the nanotubes mainly influences adhesion and viability of the tissue, whereas surface roughness also influences adhesion, but viability to a smaller extent.

The wall thickness of an individual nanotube contained in the nanotube array is preferably 5-45 nm.

After production of the nanotube array, deposits from the production procedure (debris) can be removed from the nanotube surface of the open nanotube ends. Such debris can smoothen out the surface of the tube array by filling gaps between individual tubes (particularly in the triple point between three tubes) and by partially closing the top openings of a portion of the tubes (such nanotube arrays are herein referred to as “compact nanotube array” and are depicted schematically in FIG. 4A). Debris removal yields porous nanotube structures (FIG. 4B). For such a cleaning procedure, preferably treatments with ultrasound, preferably treatment in an aqueous liquid in a ultrasound bath is employed. For titanium dioxide nanotube arrays, such deposits are titanium hydroxide and/or titanium dioxide which close the gaps between the nanotubes (FIG. 4A) (Wang 2009). By employing the described cleaning treatment, the deposits are removed and the nanotube array becomes more porous (see FIG. 4B). Such cleaning can be employed before the tissue is placed on the nanotube array to change surface properties of the nanotube array and thus, adhesion of the tissue to the nanotube array.

For application in a method or use according to the invention a nanotube array is produced by methods known in the art, a metal oxide nanotube array is preferably produced by anodisation of a metallic substrate. Therefore, a metallic sheet, on which the metal oxide nanotubes array is formed, is used as working electrode.

The nanotube array used in the invention preferably does not contain layers of silicon-containing oxides on its surface (however nanotube arrays of silicone or silicone oxides are preferably employed). Further, the nanotube array preferably does not contain DNA-binding proteins, in particular no transcription factors.

Prior to application in tissue culture the nanotube array is sterilized by methods known in the art, preferably heating, plasma cleaning, ion bombardment, alpha- beta- and gamma-irradiation or rinsing with ethanol (preferred). In a culturing method according to the invention the cultured tissue sample is contacted only with a thin film of cell culture medium on the surface of the nanotube array. In a culturing method according to the invention the cultured tissue sample is preferably not in contact with the cell culture medium bulk liquid. Hence, three general feeding mechanisms are preferred, each one of which uses a differently designed nanotube array substrate:

### 1. Closed Bottom Nanotube Array

This alternative is performed with nanotube arrays being attached to a fully closed solid support material. Hence, the nanotubes have openings at one side and are fully closed on the other side. For culturing tissue in a method according to the invention, a tissue sample is placed on the top side (the side comprising the openings of the nanotubes) of the nanotube array so that the tissue is not in contact with the bulk liquid of the cell culture medium but is only in contact with the thin film of cell culture medium covering the nanotube substrate. To create said thin film of cell culture medium on the surface of the nanotube substrate the nanotube substrate is contacted with the cell culture medium (preferably by contacting the bottom of the nanotube substrate with medium, e.g. by dipping, or by pipetting the medium on the nanotube substrate on the side of the tissue sample). The medium is subsequently drawn into the nanotubes by capillary forces thereby forming a thin layer of cell culture medium on the nanotube substrate. Thereby nutrients from the cell culture medium are continuously fed to the tissue sample via the self-restoring thin film of medium on the top of the nanotube substrate, as well as on all other surfaces of the nanotube array.

Metal oxide nanotube substrates applicable in this feeding mechanism are preferably produced by one of the following alternatives:

After production of a metal oxide nanotube array by methods known in the art, a metal oxide nanotube array is attached to both sides the initial metallic substrate (top and bottom). A so-formed metal oxide nanotube array is either used directly (with top and bottom nanotubes) or the nanotube array formed at one side is removed, preferably by etching (e.g. by treatment in water-free CH₃OH/Br₂ solution, HF acid, by ion etching or mechanical polishing), yielding a nanotube array being attached to only one side of the initial metallic substrate. The metal oxide nanotube arrays applicable in this feeding mechanism exhibit a high rigidity and hence are easy to handle. The closed bottom metal oxide nanotube array atop a metal substrate is robust and even deformable and allows for attachment of the closed bottom metal oxide nanotube array to external devices, e.g. actuators or motors to determine the effect of deformations of the tissue.

Alternatively the metal oxide nanotube array is removed from the metallic substrate (yielding a free standing nanotube array). Detachment of the metal oxide nanotube array from the initial metallic substrate is preferably employed by drying the metal oxide nanotube array substrate after anodization, preferably for several hours at 20-60° C., which automatically removes the nanotube array from the initial metallic substrate. Additionally, if the nanotubes do not detach from the initial substrate, the nanotube array can be placed in an ultrasonic bath with a liquid, preferably water or ethanol. By that the nanotubes remain closed at the ends which were initially attached to the initial metal substrate, leading to a closed bottom nanotube array.

### 2. Partially Closed Bottom Nanotube Array

This alternative is performed with nanotube arrays being attached to a solid, preferably metallic, support material, the solid support material comprising pores (openings in the support material allowing liquid transport through the pores to the other side of the support material), preferably pores of an average diameter of 100 nm-50 μm, particularly preferred 100 nm-10 μm. Hence, the nanotubes have openings at one side (top side) and are attached to the porous solid support on the other side (bottom side). For culturing tissue in a method according to the invention, a tissue sample is placed on the top side of the nanotube array. Cell culture medium is fed to the tissue as described in 1 or by contacting the nanotube array with cell culture medium on the bottom of the solid support. If cell culture medium is supplied from the bottom of the solid support, it subsequently perfuses through the pores and draws over the nanotube surface by capillary forces. The medium is subsequently forming a thin layer of cell culture medium on the nanotube substrate.

The partially closed bottom nanotube array allows a more homogenous feeding of the tissue by continuously feeding cell culture medium through the bottom of the nanotube array.

Metal oxide nanotube substrates applicable in this feeding mechanism are preferably produced by one of the following alternatives:
(i) After production of a metal oxide nanotube array by methods known in the art, a metal oxide nanotube array is attached to both sides of the initial metallic substrate (top and bottom). A so-formed metal oxide nanotube array is either used directly (with top and bottom nanotubes) or the nanotube array formed at one side of the metallic substrate is removed, preferably by etching (e.g. by treatment in water-free CH₃OH/Br₂ solution or HF acid), laser structuring, lithography or mechanical drilling yielding a nanotube array being attached to only one side of the initial metallic substrate. Pores are introduced into the metallic substrate by methods known in the art, preferably by means of focused ion beam, laser structuring, laser lithography or mechanical drilling.
(ii) Alternatively, a partially closed bottom nanotube array can be produced by coating a porous metallic substrate (preferably pores of an average diameter of 100 nm-50 μm, particularly preferred 100 nm-10 μm, metallic substrates as preferred above) with a metallic layer, preferably titanium, employing physical vapor deposition, sputter deposition or pulsed laser deposition followed by subsequent anodization as described in 1) to yield a metal oxide nanotube array with a porous substrate material on the nanotube bottom.
(iii) The metal oxide nanotube array is removed from the metallic substrate as described in 1), yielding a free standing nanotube array with closed bottom ends of the nanotubes into which pores are introduced by methods known in the art, preferably by means of focused ion beam, laser structuring, lithography or mechanical drilling.
(iv) Free-standing nanotube arrays with nanotubes open on both ends are produced. Here the nanotubes are removed from the initial metallic support material by methods described in 1) which yield nanotubes with top ends open and bottom ends closed. Methods known in the art, preferably treating the nanotube array with ultrasound in aqueous solution or etching the closed nanotube ends are employed to open the nanotube bottom ends. The nanotube array with both nanotube ends open is then attached on top of a porous support material (preferably a metallic support material with pores of an average diameter of 100 nm-50 μm, particularly preferred 100 nm-10 μm).

For methods (i)-(iv) etching and ultrasound methods described in 1) can additionally be employed to open a channel all the way from the porous support to the nanotube surface.

Preferably the nanotube array is arranged on a porous support material, preferably a grid, for culturing of a tissue sample to allow easier adjustment of the height of the cell culture medium bulk liquid.

### 3. Free Standing Open Nanotube Array

This alternative is performed with nanotube arrays that have openings at both of their ends (top and bottom side) and that are not attached to a solid support at the top and bottom side (named herein “free-standing open nanotubes”). For culturing tissue in a method according to the invention, a tissue sample is placed on the top side of the nanotube array. Cell culture medium is fed to the tissue by contacting the nanotube array with cell culture medium (at the bottom and/or side) of the array (as described in 1 and 2). Preferably the nanotube array is dipped into the cell culture medium, the cell culture medium bulk liquid being in contact with the bottom of the nanotubes, the bulk liquid not fully (preferably not at all) covering the top side of the nanotubes. The cell culture medium is drawn into the nanotubes by capillary forces thereby forming a thin layer of cell culture medium on the surface of the nanotube substrate.

The nanotube arrays applicable in this feeding mechanism exhibit less rigidity than the nanotube array substrates of the previously described feeding mechanisms 1 and 2 but the array is still more rigid than other organic substrates. Rigidity of the free standing nanotube array can however be improved by using larger nanotube lengths, preferably more than 100 μm. With free standing nanotube arrays the medium can be fed continuously and homogenously over the entire tissue surface.

Metal oxide nanotube substrates applicable in this feeding mechanism are preferably produced as follows:

After production of a metal oxide nanotube array by methods known in the art, a metal oxide nanotube array is attached to both sides the initial metallic substrate (top and bottom). The nanotube array formed on one side of the metallic substrate is lifted-off from the metallic substrate by ultrasonic treatments or by dissolving the substrate and the nanotubes on the opposite side (as described above), yielding a free-standing open metal oxide nanotube array.

Depending on the application, attachment of the cells of the cultured organ tissue to the nanotube substrate can be advantageous, e.g. when mechanical property measurements of cultured tissue are to be performed. In one embodiment of the invention the surface of the nanotube array is functionalized with bioactive substances, preferably substances that improve attachment of the organ tissue to the nanotube substrate, particularly preferred selected from poly-d-lysin, poly-L-lysin, collagen, fibronectin and laminin.

Vessels applicable for cell and tissue culture are known in the art and are applicable for tissue culture in the invention in combination with a nanotube array as defined above. Preferred are plastic vessels, preferably vessels from polystyrene, in the form of bottles, flaks or dishes. Culture media used in tissue culture in the invention are standard cell culture media known in the art for the cultivation of the respective tissue. In a tissue culture vessel according to the invention the nanotube carrier substrate is preferably arranged horizontally within the outer vessel. Preferably the nanotube carrier substrate is arranged on a porous support material, preferably a grid (preferably a metallic or plastic grid), to allow an easier control of the level of cell culture medium contacting the nanotube carrier substrate.

The invention provides vessels, uses and methods for tissue culture, preferably primary tissue culture, particularly preferred organ culture, which is to be distinguished from standard cell culture (where isolated cells are cultured in adherent or suspension culture) and tissue engineering (where cells are cultured on a three-dimensional matrix to differentiate and form a tissue or organ). Tissue to be cultured with the invention is explanted from a subject, preferably a mammal, and subsequently cultured in vitro. The invention is preferred for in vitro culture of brain, retinal, skin, liver, kidney, lung or heart tissue, preferably brain or retinal tissue. Preferably the cultured tissue is adult tissue, preferably adult mammalian tissue.

For retinal tissue culture the following nanotube array parameters are preferred (individually and in combination with each other): inner tube diameter 20-120 nm (preferably 60-100 nm), wall thickness 5-30 nm, surface roughness 5-100 nm. For brain tissue culture the following nanotube array parameters are preferred (individually and in combination with each other): inner tube diameter 10-120 nm, wall thickness 20-40 nm, surface roughness 1-50 nm.

The invention is further illustrated by the following figures and examples without being limited to these.

FIG. 1 Adult guinea pig retina cultured on a titanium dioxide nanotube array. A) Top view of a titanium dioxide nanotube array (recorded by SEM). The nanotubes exhibit an inner diameter of 80 nm (average) and a wall thickness of 10-15 nm. This substrate exhibits a surface roughness of 50-100 nm and is applicable for in vitro culture of mammalian adult retina tissue. B) Adult guinea pig retina cultured on the nanotube substrate of A) for 7 days. After culture, the retina was removed from the substrate and indirectly immunostained for nuclei (top left), photo receptors (top right), Müller cells (retina glial cells; bottom left). An overlay of the stainings is shown in the image at bottom right side.

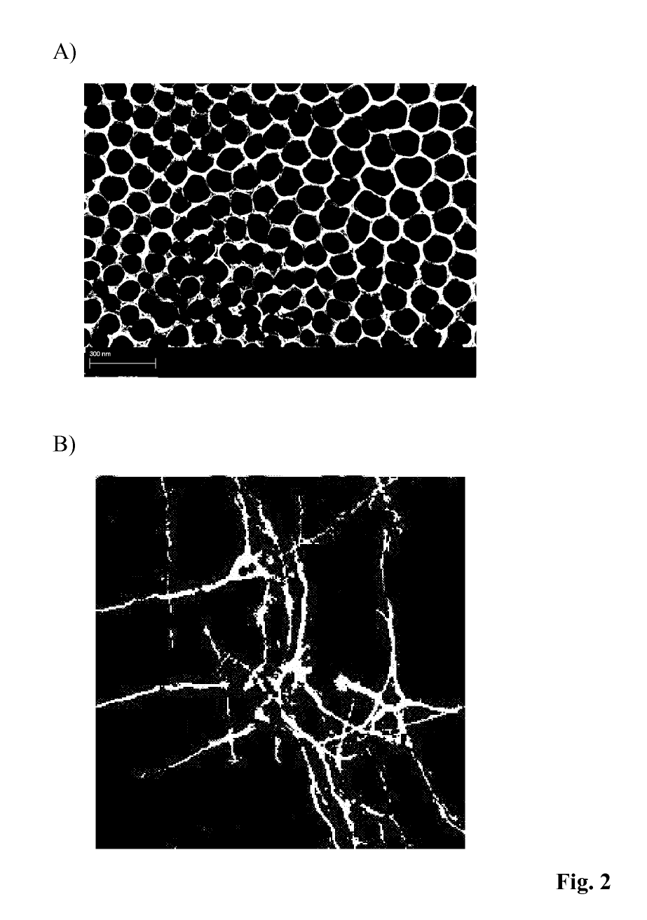

FIG. 2 Adult mouse brain slice cultured on a titanium dioxide nanotube array. A) Top view of a titanium dioxide nanotube array (recorded by SEM). The nanotubes exhibit an inner diameter of 80 nm (average) and a wall thickness of 35-40 nm. This substrate exhibits a surface roughness of 5-10 nm and is applicable for in vitro culture of mammalian adult brain tissue. B) Image of immunostained murine brain slice cultured on the titanium nanotube array according to A) for six days indirectly stained with neurofilament marker SMI32 to image the neurofilaments of the neuronal cells.

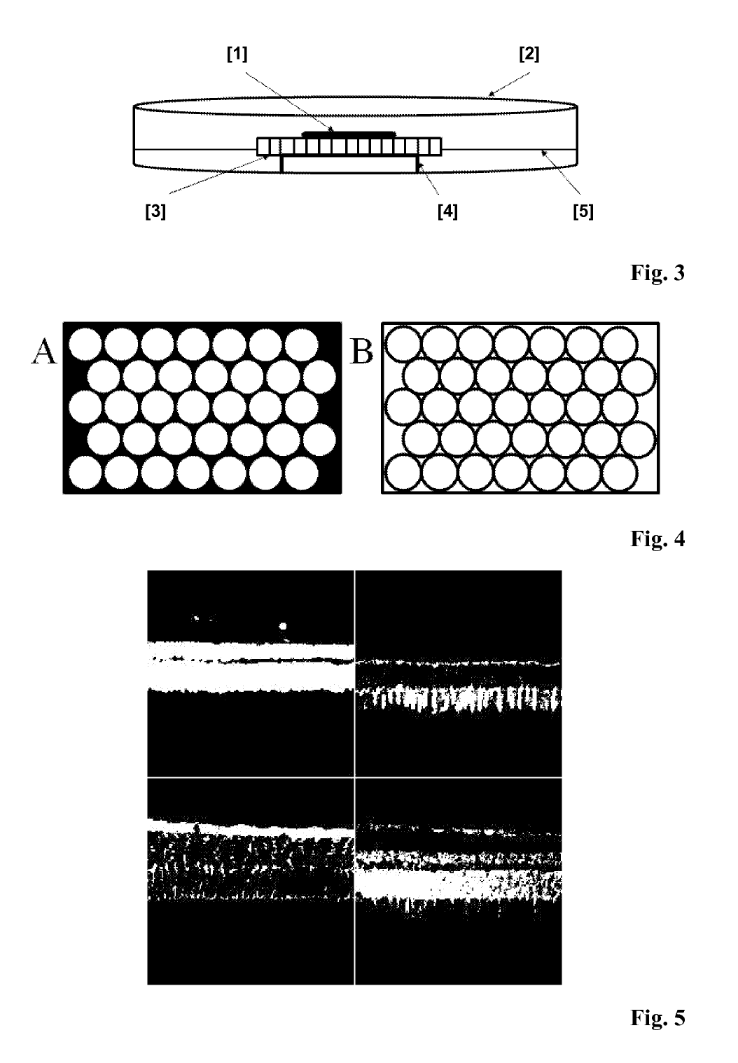

FIG. 3 Illustration of a cell culture vessel including a nanotube carrier substrate in its application. The nanotube carrier substrate (nanotube array, [3]) is located in an outer cell culture dish [2] and (optionally) placed on a grid [4]. Cell culture medium [5] is filled into the cell culture dish to contact the nanotube carrier substrate at the bottom and sides (arrow indicates the height of the cell culture medium inside the cell culture dish). The nanotube carrier substrate sticks out of the cell culture medium bulk liquid and the tissue sample [1] is located on the top side of the nanotube substrate. A thin self-restoring film of cell culture medium is drawn onto the surface of the nanotube carrier substrate thereby feeding the tissue sample with cell culture medium.

FIG. 4 Schematic drawing of nanotube arrays (top view). A) compact nanotube array. B) porous nanotube array obtainable by ultrasound treatment of a compact nanotube array. Black areas indicate solid material (e.g. titanium dioxide), white areas indicate pores.

FIG. 5 Confocal laser scanning microscopy image of a freshly extracted guinea pig retina. The retina was immunostained for nuclei (top left), photo receptors (top right), Müller cells (retina glial cells; bottom left). An overlay of the stainings is shown in the image at bottom right side.

### EXAMPLE 1

### Cultivation of an Adult Guinea Pig Retina In Vitro

Adult guinea pig retina was explanted and cultured on a titanium dioxide nanotube array as shown in FIG. 1A that exhibited a surface roughness of 50-100 nm and an average nanotube diameter of 80 nm The nanotube substrate was chosen in the form of a closed bottom nanotube array (where the nanotubes were grown on a continuous titanium plate), deposited on a grid inside an outer cell culture vessel (commercially available cell culture dish) as illustrated in FIG. 3. A nanotube substrate with closed tubes on both sides of the titanium plate was used.

Adult guinea pig retina was isolated from eyes of adult guinea pig. After enucleation of the eyes, they were transferred into Ames cell culture medium (Sigma-Aldrich:A1420) without any supplements at 4° C. and were processed under aseptic conditions. The eyes were quickly rinsed in ethanol 70% (v/v), washed in PBS and kept in the Ames medium without supplements. An incision was made between cornea and sclera to lower down the pressure and remove the lens and the vitreous body. At this point the retina was gently removed from the eye cup and cut in four pieces. The retinal pieces were flat-mounted with the photoreceptor-side down on the nanotube substrate. The nanotube substrate was placed on the grid inside a cell culture dish which was filled with cell culture medium of a height to touch the nanotube substrate bottom and sides. However, the top side of the nanotube substrate and the retina tissue were not covered by culture medium. Nutrient supply from the cell culture medium was assured by perfusion of the medium up the side walls of the nanotube substrate by capillary forces to the bottom side of the tissue sample. The culture medium (Ames Medium, 10% Horse Serum, 0.1% Gentamicin) was changed the day after isolation and then every third day. It is known that adult retinal tissue gets destroyed and dies when covered with liquid medium during the duration of in vitro culture on different organ culture substrates. Surprisingly, although being cultured on a closed bottom nanotube array fed with cell culture medium by pipetting it to the side ends of the retina instead of perfusion through the nanotubes from the bottom the retina was viable for up to 14 days.

For immunohistochemical staining the isolated and cultured retinas (after 7 days of culture) were fixed after culture in 4% paraformalaldehyde overnight at 4° C. After washing in PSB the tissues were embedded in PSB contain 3% agarose (Sigma-Aldrich Fluka:05073), and then 50 μm-thick slices were cut with a vibration microtome (HM 650 V; Microm-International, Walldorf, Germany). The slices were incubated in 5% normal goat serum and 0.3% Triton, 1% DMSO in PBS (PSB-TD) for 1 h at 5° C., then washed with PBS-TD. The slices were exposed overnight at 4° C. to monoclonal AntiVimentin Clone 9 (Sigma-Aldrich V6630) and peanut agglutinin (PNA) (Sigma-Aldrich L6135). The day after the slices were washed with PBS-TD for 3 hours on the shaker changing the PBS-TD every hour, then exposed to Cy2-conjugated streptavidin and Cy3-conjugated goat-antimouse (Jackson Immunoresrearch) overnight at 4° C. The third day the slices were washed with PBS-TD for 1 hour on the shaker, then incubated with TO-PRO-3-iodide (Invitrogen: T3605) for 1 hour at room temperature. The lastx step is washing the slices with normal PBS and then put them on a coverslip coated with IMMUNOMOUNT. Confocal imaging was carried using an upright laser scanning microscope (Zeiss 510 META, Jena, Germany) equipped with an enterprise laser.

A confocal laser scanning microscopy image of the retina after 7 days of culture is shown in FIG. 1B. The image shows the intact and viable structure of the retina with the healthy Müller cells and photoreceptors. For comparison, FIG. 5 shows a confocal laser scanning microscopy image of a freshly extracted guinea pig retina using identical markers and staining procedure. As can be seen in FIG. 1B an organotypic culture of the adult retina was obtained. The cultured organ tissue could be detached from the substrate for later analysis without disruption.

In another experiment, an adult guinea pig retina was cultured under the same conditions on a nanotube as shown in FIG. 1A, which was coated with poly-d-lysin before the retina was placed on it. Coating of the nanotube array with poly-d-lysin improved physiological conditions and increased the adhesion of the retina to the nanotube substrate.

### EXAMPLE 2

### Cultivation of an Adult Murine Brain Slices In Vitro

Brain slices from adult mice were cultured on a titanium dioxide nanotube array as shown in FIG. 2A that exhibited a surface roughness of 5-10 nm and an average nanotube diameter of 80 nm. The nanotube substrate was chosen in the form of a closed bottom nanotube array (where the nanotubes were grown on a continuous titanium plate). The nanotube substrate was chosen in the form of a closed bottom nanotube array (where the nanotubes were grown on a continuous titanium plate), deposited on a grid inside an outer cell culture vessel (commercially available cell culture dish) as illustrated in FIG. 3. A nanotube substrate with closed tubes on both sides of the titanium plate was used.

Brain was extracted from adult mice, cut into slices of about 100-250 μm thickness and placed onto the top of a titanium dioxide nanotube array as shown in FIG. 2A. The cells were cultured in Hams DMEM medium with L-Glutamin, 24% horse serum, 1% Glucose, 2% HEPES, 0.1% gentamycin (GIBCO) (other conditions being equal as described in example 1). After 6 days of in vitro culture the neuronal cells were immunostained with SMI32-Cy3 (neurofilaments) (FIG. 2B). The neurons within the brain slice remained viable and organotypic over the whole culture period.

Cited Non Patent Literature

| | |
|---|---|
| Caffe 2001 | Caffé A R, Ahuja P, Holmqvist B, Azadi S, Forsell J, Holmqvist I, |
| | Söderpalm A K, van Veen T.: J Chem Neuroanat. 2001 November; 22(4): 263-73. |
| Desai et al. 1999 | T. A. Desai, J. Deutsch, D. Motlagh, W. Tan and B. Russell, Biomed. |
| | Microdev. 2, 123 (1999) |
| Eschbach 2005 | E. Eschbach, S. S. Chatterjee, M. Nöldner, E. Gottwald, H. Dertinger, K.- |
| | F. Weibezahn and G. Knedlitschek, Journal of Cellular Biochemistry 95, |
| | 243 (2005) |
| Feigenspan 1993 | Feigenspan A, Bormann J, Wässle H.: Vis Neurosci. 1993 March- |
| | April; 10(2): 203-17. |
| Germer 1997 | Germer A, Jahnke C, Mack A, Enzmann V, Reichenbach A.: Neuroreport. |
| | 1997 Sep. 29; 8(14): 3067-72. |
| Giselbrecht 2006 | S. Giselbrecht, T. Gietzelt, E. Gottwald, C. Trautmann, R. Truckenmüller, |
| | K. F. Weibezahn and A. Welle, Biomed. Microdev. 8, 191 (2006 |
| Johansson 2005 | Johansson K, Ehinger B.: Vision Res. 2005 November; 45(25-26): 3235-43. Epub |
| | 2005 Jul. 15. |
| Kaempf 2008 | Kaempf S, Walter P, Salz A K, Thumann G.: J Neurosci Methods. 2008 |
| | Aug. 15; 173(1): 47-58. Epub 2008 Jul. 15. |
| Kobuch 2008 | Kobuch K, Herrmann W A, Framme C, Sachs H G, Gabel V P, Hillenkamp |
| | J.: Exp Eye Res. 2008 April; 86(4): 661-8. Epub 2008 Jan. 18. |
| Koizumi 2007 | Koizumi, G. Zeck, Y. Ben, R. H. Masland and T. C. Jacobs, Plos ONE 2, |
| | e221 (2007) |
| Kuhrt 2008 | Kuhrt H, Wurm A, Karl A, Iandiev I, Wiedemann P, Reichenbach A, |
| | Bringmann A, Pannicke T.: Int J Dev Neurosci. 2008 November; 26(7): 745-51. |
| | Epub 2008 Jul. 11. |
| Kunz-Schughart 2004 | L. A. Kunz-Schughart, J. P. Freyer, F. Hofstaedter and R. Ebner, J. |
| | Biomol. Screen. 9, 273 (2004) |
| Leclerc 2003 | E. Leclerc, Y. Sakai and T. Fujii, Biomed. Microdev. 5, 209 (2003) |
| Ogilvie 1999 | Ogilvie J M, Speck J D, Lett J M, Fleming T T.: J Neurosci Methods. 1999 |
| | Feb. 1; 87(1): 57-65. |
| Roy 2011 | Roy P, Berger S, Schmuki P.: Angew Chem Int Ed Engl. 2011 Mar. |
| | 21; 50(13): 2904-39. doi: 10.1002/anie.201001374. Epub 2011 Mar. 10. |
| | Review |
| Rzeczinski 2006 | Rzeczinski S, Victorov IV, Lyjin A A, Aleksandrova O P, Harms C, |
| | Kronenberg G, Freyer D, Scheibe F, Priller J, Endres M, Dirnagl U.: |
| | Ophthalmic Res. 2006; 38(5): 263-9. Epub 2006 Sep. 15. |
| Snyder 2001 | J. D. Snyder and T. A. Desai, J. Biomater. Sci. Polym Ed. 12, 921 (2001) |
| Spencer 2008 | N. J. Spencer, D. A. Cotanche and C. M. Klapperich, Biomaterials 29, |
| | 1028 (2008) |
| Wang 2009 | Chemistry of Materials 21(7), 1198-1206 (2009) |
| Xin 2007 | Xin H, Yannazzo J A, Duncan R S, Gregg E V, Singh M, Koulen P.: J |
| | Neurosci Methods. 2007 Jan. 15; 159(1): 35-42. Epub 2006 Jul. 31. |
| Yamauchi 2003 | N. Yamauchi, O. Yamada, T. Takahashi, K. Imai, T. Sato, A. Ito and K. |
| | Hashizume, Placenta 24, 258 (2003) |

What is claimed is:

1. Tissue culture vessel, comprising an outer vessel and a nanotube carrier substrate located within the outer vessel, wherein the nanotube carrier substrate comprises a nanotube array having a surface roughness of 1 nm to 100 nm, wherein the nanotube array is horizontally arranged inside the outer vessel and comprises an upper side and a bottom side, the upper side comprising openings of the nanotubes and the bottom side being attached to a solid support material containing pores of a diameter of 100 nm to 50 μm.

2. (canceled)

3. (canceled)

4. Method for in vitro culturing primary tissue, preferably primary mammalian tissue, wherein
a nanotube array, having a surface roughness of 1 nm to 100 nm and comprising an upper side and a bottom side, the upper side comprising openings of the nanotubes is horizontally arranged inside an outer tissue culture vessel, so that openings of the nanotubes point at least in upward direction and the bottom side is attached to a solid support material containing pores of a diameter of 100 nm to 50 μm,
the nanotube array is contacted with cell culture medium,
an isolated primary tissue sample is placed on said nanotube array.

5. Method of claim 4, wherein the upper surface of said nanotube array sticks out of the cell culture medium bulk liquid or is adjusted at the same height as the cell culture medium bulk liquid surface.

6. Tissue culture vessel of claim 1, wherein the nanotube array is a metal oxide nanotube array or a carbon nanotube array, preferably a metal oxide nanotube array, particularly preferred from nanotubes of oxides of titanium, zirconium, hafnium, silicon, aluminum, gold, silver, copper, platinum, vanadium, palladium, and/or niobium, preferably titanium oxide.

7. (canceled)

8. Tissue culture vessel of claim 1, wherein the bottom side is closed.

9. (canceled)

10. Tissue culture vessel of claim 1, wherein the upper side and the bottom side comprise openings of the nanotubes.

11. Tissue culture vessel of claim 1, wherein the nanotube array is obtainable by electrolytic anodisation using a metallic sheet, on which a metal oxide nanotube array is formed, as working electrode.

12. Tissue culture vessel of claim 1, wherein the nanotube array comprises a surface coating comprising at least one bioactive substance, preferably selected from poly-d-lysin, poly-L-lysin, collagen, fibronectin and laminin.

13. (canceled)

14. (canceled)

15. (canceled)

16. (canceled)

17. (canceled)

18. (canceled)

19. (canceled)

20. Method of claim 4, wherein the nanotube array is a metal oxide nanotube array or a carbon nanotube array, preferably a metal oxide nanotube array, particularly preferred from nanotubes of oxides of titanium, zirconium, hafnium, silicon, aluminum, gold, silver, copper, platinum, vanadium, palladium, and/or niobium, preferably titanium oxide.

21. (canceled)

22. Method of claim 4, wherein the bottom side is closed.

23. (canceled)

24. Method of claim 4, wherein the upper side and the bottom side comprise openings of the nanotubes.

25. Method of claim 4, wherein the nanotube array is obtainable by electrolytic anodisation using a metallic sheet, on which a metal oxide nanotube array is formed, as working electrode.

26. Method of claim 4, wherein the nanotube array comprises a surface coating comprising at least one bioactive substance, preferably selected from poly-d-lysin, poly-L-lysin, collagen, fibronectin and laminin.
